(19) United States
(12) Patent Application Publication
(10) Pub. No.: US 2026/0125327 A1
(43) Pub. Date: May 7, 2026

(54) METHOD TO FABRICATE MULTIPHASE ZIRCONIA MATERIAL TO ACHIEVE HIGH FRACTURE TOUGHNESS

(71) Applicant: James R. Glidewell Dental Ceramics, Inc., Newport Beach, CA (US)

(72) Inventors: Dimple Pradhan, Foothill Ranch, CA (US); Yan Yang, Irvine, CA (US); Sreeram Balasubramanian, Irvine, CA (US)

(73) Assignee: James R. Glidewell Dental Ceramics, Inc., Newport Beach, CA (US)

(21) Appl. No.: 18/953,672

(22) Filed: Nov. 20, 2024

Related U.S. Application Data
(60) Provisional application No. 63/602,008, filed on Nov. 22, 2023.

(51) Int. Cl. C04B 35/486; C04B 35/64

(52) U.S. Cl. CPC C04B 35/486; C04B 35/64; C04B 2235/3225; C04B 2235/3246; C04B 2235/96

(57) ABSTRACT

Yttria-stabilized zirconia ceramic bodies with a composite system comprising two or more phases. The sintered yttria-stabilized zirconia ceramic bodies have localized regions with different yttria concentrations within the composite system. These microregions containing varying yttria concentrations result in a non-uniform distribution of yttria ranges which individually contribute to localized characteristics that synergistically provide improved fracture toughness and machinability.

This application claims the benefit of and priority to U.S. Provisional Patent Application No. 63/602,008, filed Nov. 22, 2023, which is incorporated herein by reference in its entirety.

### BACKGROUND

Yttria-stabilized zirconia (YSZ) has been an attractive material for dental applications due to an excellent combination of properties-esthetics, chemical resistance, mechanical strength and toughness, and biocompatibility. Better esthetics (translucency) and mechanical properties are the key factors to provide the patient with more reliable and natural looking dental restorations. Typically, tetragonal YSZ phase (low-Y₂O₃ level) provides excellent mechanical properties while cubic YSZ phase (high-Y₂O₃ level) makes the material more translucent. Currently, most of the existing dental grade zirconia materials have a certain Y₂O₃ level resulting in a uniform microstructure, which tends to compromise on either mechanical properties or translucency.

### SUMMARY

Disclosed herein are yttria-stabilized zirconia ceramic bodies with a composite system comprising two or more phases. The sintered yttria-stabilized zirconia ceramic bodies have localized regions with different yttria concentrations within the composite system. These microregions containing varying yttria concentrations result in a non-uniform distribution of yttria ranges which individually contribute to localized characteristics that synergistically provide improved fracture toughness and machinability.

Also disclosed herein are sintered yttria-stabilized zirconia ceramic bodies comprising a composite system with different localized yttria concentrations within the composite system, wherein more than 55% of the localized yttria concentrations have a yttria concentration of less than 2.5 mol %, at least 10% of the localized yttria concentrations have a yttria concentration between 4 and 5.5 mol %, and at least 90% of the localized yttria concentrations have a yttria concentration of less than 5 mol %, wherein the sintered yttria-stabilized zirconia body has a fracture toughness of greater than 8, and the mol % is based on the total moles of the yttria-stabilized zirconia at the respective location. In some embodiments of the foregoing ceramic bodies, 100% of the localized yttria concentrations have a yttria concentration of less than 5.5 mol %. In some further embodiments, the ceramic bodies have a bulk yttria concentration of 2.8 mol % to 3.1 mol %. In some further embodiments, the ceramic bodies are dental prosthetic devices.

Also disclosed herein are sintered yttria-stabilized zirconia bodies comprising a composite system with different localized yttria concentrations within the composite system, wherein more than 45% of the localized yttria concentrations have a yttria concentration of less than 2.5 mol %, at least 30% of the localized yttria concentrations have a yttria concentration of between 4 and 5.5 mol %, more than 60% of the localized yttria concentrations have a yttria concentration of less than 2.8 mol %, and at least 95% of the localized yttria concentrations have a yttria concentration of less than 5 mol %, wherein the sintered yttria-stabilized zirconia ceramic body has a fracture toughness of greater than 6.8, and the mol % is based on the total moles of the yttria-stabilized zirconia at the respective location. In some embodiments of the foregoing ceramic bodies, 100% of the localized yttria concentrations have a yttria concentration of less than 5.5 mol %. In some further embodiments, the ceramic bodies have a bulk yttria concentration of 3.2 mol % to 3.5 mol %. In some further embodiments, the ceramic bodies are dental prosthetic devices.

Also disclosed herein are sintered yttria-stabilized zirconia bodies comprising a composite system with different localized yttria concentrations within the composite system, wherein more than 20% of the localized yttria concentrations have a yttria concentration of less than 2.5 mol %, at least 60% of the localized yttria concentrations have a yttria concentration of between 4 and 5.5 mol %, and at least 60% of the localized yttria concentrations have a yttria concentration of less than 5 mol %, wherein the sintered yttria-stabilized zirconia ceramic body has a fracture toughness of greater than 5, and the mol % is based on the total moles of the yttria-stabilized zirconia at the respective location. In some embodiments of the foregoing ceramic bodies, 100% of the localized yttria concentrations have a yttria concentration of less than 5.5 mol %. In some further embodiments, the ceramic bodies have a bulk yttria concentration of 3.6 mol % to 4 mol %. In some further embodiments, the ceramic bodies are dental prosthetic devices.

Also disclosed herein are sintered yttria-stabilized zirconia bodies comprising a composite system with different localized yttria concentrations within the composite system, wherein more than 10% of the localized yttria concentrations have a yttria concentration of less than 2.5 mol %, at least 80% of the localized yttria concentrations have a yttria concentration of between 4 and 5.5 mol %, and at least 50% of the localized yttria concentrations have a yttria concentration of less than 5 mol %, wherein the sintered yttria-stabilized zirconia ceramic body has a fracture toughness of greater than 4, and the mol % is based on the total moles of the yttria-stabilized zirconia at the respective location. In some embodiments of the foregoing ceramic bodies, 100% of the localized yttria concentrations have a yttria concentration of less than 5.5 mol %. In some further embodiments, the ceramic bodies have a bulk yttria concentration of 4 mol % to 4.5 mol %. In some further embodiments, the ceramic bodies are dental prosthetic devices.

Also disclosed herein are sintered yttria-stabilized zirconia ceramic bodies comprising a composite system with different localized yttria concentrations within the composite system, wherein more than 80% of the localized yttria concentrations have a yttria concentration of less than 2.5 mol %, less than 10% of the localized yttria concentrations have a yttria concentration greater than 5.8 mol %, less than 5% of the localized yttria concentrations have a yttria concentration greater than 6.5 mol %, and at least 10% of the localized yttria concentrations have a yttria concentration of between 6 and 7.5 mol %, wherein the sintered yttria-stabilized zirconia ceramic body has a fracture toughness of greater than 8, and the mol % is based on the total moles of the yttria-stabilized zirconia at the respective location. In some embodiments of the foregoing ceramic bodies, 100% of the localized yttria concentrations have a yttria concentration of less than 7.5 mol %. In some further embodiments, the ceramic bodies have a bulk yttria concentration of 2.8 mol % to 3.1 mol %. In some further embodiments, the ceramic bodies are dental prosthetic devices.

Also disclosed herein are sintered yttria-stabilized zirconia ceramic bodies comprising a composite system with different localized yttria concentrations within the composite system, wherein more than 50% of the localized yttria concentrations have a yttria concentration of less than 2.5 mol %, more than 55% of the localized yttria concentrations have a yttria concentration of less than 2.8 mol %, less than 40% of the localized yttria concentrations have a yttria concentration greater than 5.8 mol %, less than 30% of the localized yttria concentrations have a yttria concentration greater than 6.5 mol %, and at least 30% of the localized yttria concentrations have a yttria concentration of between 6 and 7.5 mol %, wherein the sintered yttria-stabilized zirconia ceramic body has a fracture toughness of greater than 5, and the wt % is based on the total weight of the yttria-stabilized zirconia at the respective location. In some embodiments of the foregoing ceramic bodies, the sintered yttria-stabilized zirconia ceramic bodies have a fracture toughness of greater than 6.8. In some further embodiments of the foregoing ceramic bodies, 100% of the localized yttria concentrations have a yttria concentration of less than 7.5 mol %. In still further embodiments, the ceramic bodies have a bulk yttria concentration of 3.2 wt % to 3.5 mol %. In still further embodiments, the ceramic bodies have a bulk yttria concentration of 3.6 wt % to 4 mol %. In some further embodiments, the ceramic bodies are dental prosthetic devices.

Also disclosed herein are sintered yttria-stabilized zirconia ceramic bodies comprising a composite system with different localized yttria concentrations within the composite system, wherein more than 30% of the localized yttria concentrations have a yttria concentration of less than 2.5 mol %, less than 50% of the localized yttria concentrations have a yttria concentration greater than 5.8 mol %, less than 50% of the localized yttria concentrations have a yttria concentration greater than 6.5 mol %, and at least 35% of the localized yttria concentrations have a yttria concentration of between 6 and 7.5 mol % wherein the sintered yttria-stabilized zirconia body has a fracture toughness of greater than 4, and the mol % is based on the total moles of the yttria-stabilized zirconia at the respective location. In some embodiments of the foregoing ceramic bodies, 100% of the localized yttria concentrations have a yttria concentration of less than 7.5 mol %. In some further embodiments, the ceramic bodies have a bulk yttria concentration of 4 mol % to 4.7 mol %. In some further embodiments, the ceramic bodies are dental prosthetic devices.

Also disclosed herein are sintered yttria-stabilized zirconia ceramic bodies comprising a composite system with different localized yttria concentrations within the composite system, wherein more than 20% of the localized yttria concentrations have a yttria concentration of less than 2.5 mol %, less than 55% of the localized yttria concentrations have a yttria concentration greater than 5.8 mol %, less than 50% of the localized yttria concentrations have a yttria concentration greater than 6.5 mol %, and at least 40% of the localized yttria concentrations have a yttria concentration of between 6 and 7.5 mol % wherein the sintered yttria-stabilized zirconia body has a fracture toughness of greater than 3, and the mol % is based on the total moles of the yttria-stabilized zirconia at the respective location. In some embodiments of the foregoing ceramic bodies, 85% of the localized yttria concentrations have a yttria concentration of less than 7.5 mol %. In some further embodiments, the ceramic bodies have a bulk yttria concentration of 4.7 mol % to 5.5 mol %. In some further embodiments, the ceramic bodies are dental prosthetic devices.

Also disclosed here are methods comprising:
mixing a first yttria-stabilized zirconia powder having a yttria concentration of 4 to 8 mol % yttria, based on the total weight of the first powder, with a second yttria-stabilized zirconia powder having a yttria concentration of 2 to 2.6 mol % yttria, based on the total weight of the second powder;
forming the resulting mixture into a desired shape, and
sintering the shaped mixture resulting in a yttria-stabilized zirconia ceramic body.

In some embodiments of the foregoing method, the second yttria-stabilized zirconia powder has a yttria concentration of 2 mol % yttria. In some other embodiments, the first yttria-stabilized zirconia powder has a yttria concentration of 8 mol % yttria and the second yttria-stabilized zirconia powder has a yttria concentration is 2 mol % yttria. In some other embodiments, the first yttria-stabilized zirconia powder has a yttria concentration of 5.3 mol % yttria and the second yttria-stabilized zirconia powder has a yttria concentration is 2 mol % yttria. In some other embodiments, the first yttria-stabilized zirconia powder has a yttria concentration of 4 mol % yttria and the second yttria-stabilized zirconia powder has a yttria concentration is 2 mol % yttria. In some other embodiments, 5 to 70 wt % of the first yttria-stabilized zirconia powder is mixed with 30 to 95 wt % of the second yttria-stabilized zirconia powder, based on the total weight of the mixture.

The foregoing and will become more apparent from the following detailed description, which proceeds with reference to the accompanying figures.

### BRIEF DESCRIPTION OF THE DRAWINGS

FIG. 1. Schematic diagram for pre-marked sheet containing 30 points for Energy Dispersive X-ray Spectroscopy (EDS) spot analysis on a sintered zirconia test sample.

FIG. 2. EDS comparison of yttria distribution obtained from single source powder system 3Y and composite system obtained by mixing Low Yttria (2Y) and High Yttria (8Y) powder systems at targeted Yttria level 3Y.

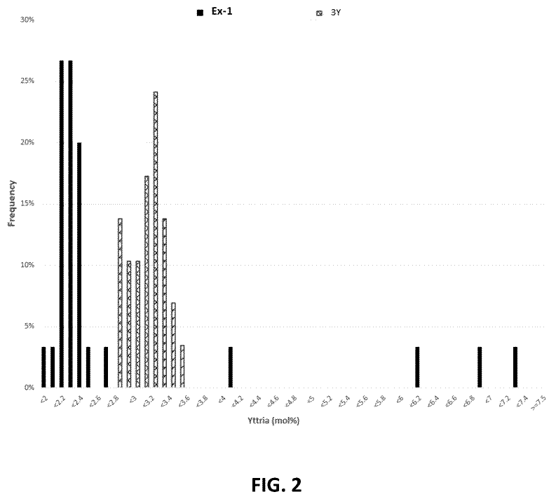

FIG. 3. Graph correlating the fracture toughness obtained from a single source powder system and mixture of Low Yttria (2Y or 3Y) and High Yttria powder systems (5.3Y or 4Y) at targeted Yttria level between 3Y-5.1Y.

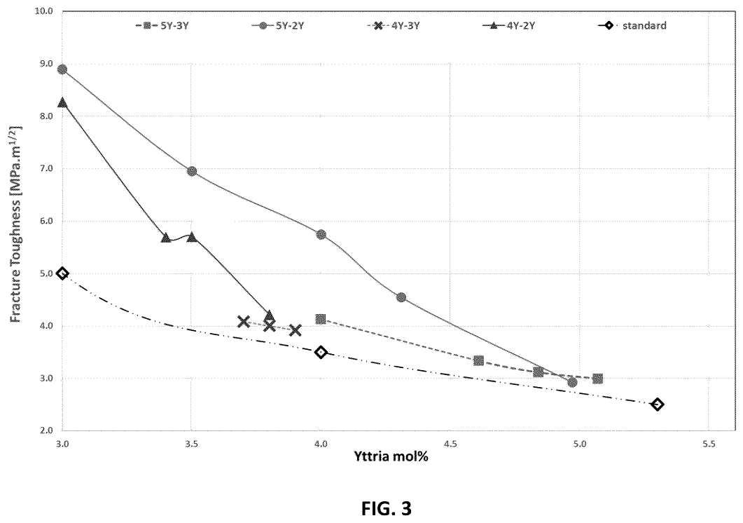

FIG. 4. Graphs correlating the fracture toughness obtained from a single source powder system and mixture of Low Yttria (2Y or 3Y) and High Yttria powder systems (8Y) at targeted Yttria level between 3Y-5.6Y.

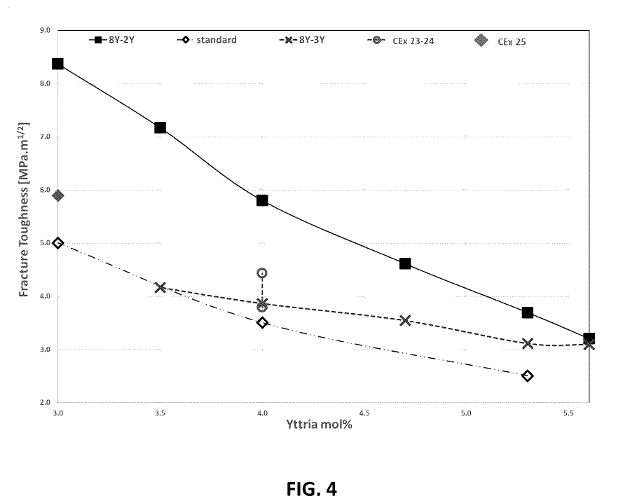

FIG. 5. Scanning electron microscope (SEM) image of microstructure of sintered YSZ body obtained from mixed powder systems.

FIG. 6. Graph illustrating the cumulative frequency of distribution of yttria level in mixed systems having Fracture toughness >8.

FIG. 7. Graph illustrating the cumulative frequency of distribution of yttria level in mixed systems having Fracture toughness >5.

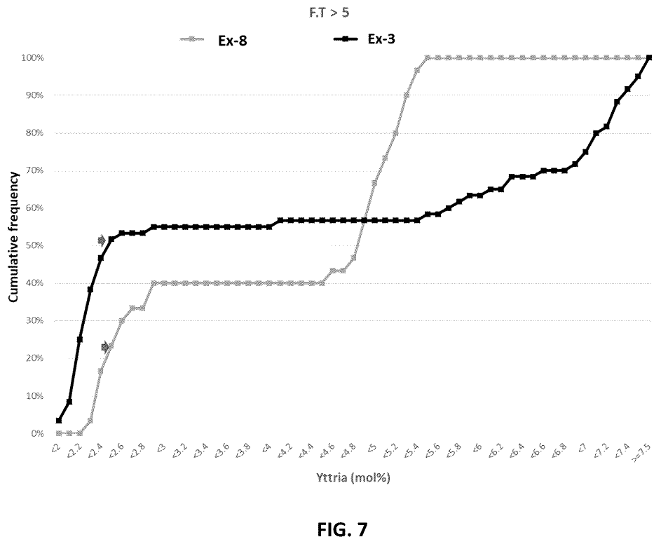

FIG. 8 Image illustrating the crack propagation in composite systems obtained by indentation method.

FIG. 9. Schematic illustrating the crack length determination after indentation samples for Fracture Toughness analysis.

FIG. 10. Graph showing the frequency of crack length distribution for mixed system having Fracture Toughness >8.

FIG. 11. Graph showing the frequency of crack length distribution for mixed system having Fracture Toughness >5.

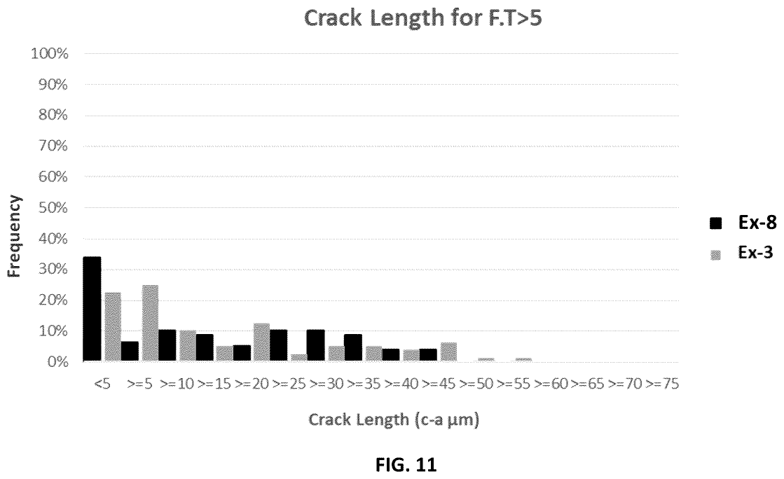

### DETAILED DESCRIPTION

YSZ crystals are arranged in crystalline cells (mesh) which can be categorized in three crystallographic phases: 1) the cubic (C) in the form of a straight prism with square sides, 2) the tetragonal (T) in the form of a straight prism with rectangular sides, and 3) the monoclinic (M) in the form of a deformed prism with parallelepiped sides. In general, tetragonal YSZ phase (low-Y₂O₃ level (i.e., less than 2Y %-3Y %)) provides excellent mechanical properties while cubic YSZ phase (high-Y₂O₃ level (i.e., more than 3Y %-8Y %)) makes the material more translucent.

Machinability is an important property of sintered YSZ bodies. For example, milling of a sintered YSZ block in a dental office is increasingly desirable. Such material must have high toughness and improved machinability. Disclosed herein are YSZ materials with a composite system comprising two or more phases that exhibit higher fracture toughness and machinability at equivalent yttria levels.

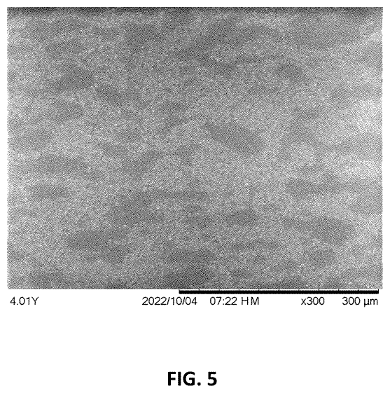

Disclosed herein are sintered yttria-stabilized zirconia ceramic bodies having localized regions with different yttria concentrations within the composite system. These microregions containing varying yttria concentrations (shown in FIG. 5) result in a non-uniform distribution of yttria ranges which individually contribute to localized characteristics that synergistically provide improved fracture toughness and machinability. For example, microregions of tetragonal clusters (localized low-Y₂O₃ level) are interspersed with microregions of cubic clusters (localized high-Y₂O₃ level). In certain embodiments, each microregion of low and high Y₂O₃ levels is less than a spherical diameter of 100 microns in size.

One embodiment is a sintered yttria-stabilized zirconia ceramic body having a composite system with different localized yttria concentrations within the composite system, wherein more than 55% of the localized yttria concentrations have a yttria concentration of less than 2.5 mol %, at least 10% of the localized yttria concentrations have a yttria concentration between 4 and 5.5 mol %, and at least 90% of the localized yttria concentrations have a yttria concentration of less than 5 mol %, wherein the sintered yttria-stabilized zirconia body has a fracture toughness of greater than 8, and the mol % is based on the total moles of the yttria-stabilized zirconia at the respective location. In certain examples of this embodiment, 100% of the localized yttria concentrations have a yttria concentration of less than 5.5 mol %. In certain examples of this embodiment, the ceramic body has a bulk yttria concentration of 2.8 mol % to 3.1 mol %.

Another embodiment is a sintered yttria-stabilized zirconia body having a composite system with different localized yttria concentrations within the composite system, wherein more than 45% of the localized yttria concentrations have a yttria concentration of less than 2.5 mol %, more than 60% of the localized yttria concentrations have a yttria concentration of less than 2.8 mol %, at least 30% of the localized yttria concentrations have a yttria concentration of between 4 and 5.5 mol %, and at least 95% of the localized yttria concentrations have a yttria concentration of less than 5 mol %, wherein the sintered yttria-stabilized zirconia ceramic body has a fracture toughness of greater than 6.8, and the mol % is based on the total moles of the yttria-stabilized zirconia at the respective location. In certain examples of this embodiment, 100% of the localized yttria concentrations have a yttria concentration of less than 5.5 mol %. In certain examples of this embodiment, the ceramic body has a bulk yttria concentration of 3.2 mol % to 3.5 mol %.

Another embodiment is a sintered yttria-stabilized zirconia body having a composite system with different localized yttria concentrations within the composite system, wherein more than 20% of the localized yttria concentrations have a yttria concentration of less than 2.5 mol %, at least 60% of the localized yttria concentrations have a yttria concentration of between 4 and 5.5 mol %, and at least 60% of the localized yttria concentrations have a yttria concentration of less than 5 mol %, wherein the sintered yttria-stabilized zirconia ceramic body has a fracture toughness of greater than 5, and the mol % is based on the total moles of the yttria-stabilized zirconia at the respective location. In certain examples of this embodiment, 100% of the localized yttria concentrations have a yttria concentration of less than 5.5 mol %. In certain examples of this embodiment, the ceramic body has a bulk yttria concentration of 3.6 mol % to 4 mol %.

Another embodiment is a sintered yttria-stabilized zirconia body having a composite system with different localized yttria concentrations within the composite system, wherein more than 10% of the localized yttria concentrations have a yttria concentration of less than 2.5 mol %, at least 80% of the localized yttria concentrations have a yttria concentration of between 4 and 5.5 mol %, and at least 50% of the localized yttria concentrations have a yttria concentration of less than 5 mol %, wherein the sintered yttria-stabilized zirconia ceramic body has a fracture toughness of greater than 4, and the mol % is based on the total moles of the yttria-stabilized zirconia at the respective location. In certain examples of this embodiment, 100% of the localized yttria concentrations have a yttria concentration of less than 5.5 mol %. In certain examples of this embodiment, the ceramic body has a bulk yttria concentration of 4 mol % to 4.5 mol %.

Another embodiment is a sintered yttria-stabilized zirconia ceramic body having a composite system with different localized yttria concentrations within the composite system, wherein more than 80% of the localized yttria concentrations have a yttria concentration of less than 2.5 mol %, less than 10% of the localized yttria concentrations have a yttria concentration greater than 5.8 mol %, less than 5% of the localized yttria concentrations have a yttria concentration greater than 6.5 mol %, and at least 10% of the localized yttria concentrations have a yttria concentration of between 6 and 7.5 mol % wherein the sintered yttria-stabilized zirconia ceramic body has a fracture toughness of greater than 8, and the mol % is based on the total moles of the yttria-stabilized zirconia at the respective location. In certain examples of this embodiment, 100% of the localized yttria concentrations have a yttria concentration of less than 7.5 mol %. In certain examples of this embodiment, the ceramic body has a bulk yttria concentration of 2.8 mol % to 3.1 mol %.

Another embodiment is a sintered yttria-stabilized zirconia ceramic body having a composite system with different localized yttria concentrations within the composite system, wherein more than 50% of the localized yttria concentrations have a yttria concentration of less than 2.5 mol %, more than 55% of the localized yttria concentrations have a yttria concentration of less than 2.8 mol %, less than 40% of the localized yttria concentrations have a yttria concentration greater than 5.8 mol %, less than 30% of the localized yttria concentrations have a yttria concentration greater than 6.5 mol %, and at least 30% of the localized yttria concentrations have a yttria concentration of between 6 and 7.5 mol %, wherein the sintered yttria-stabilized zirconia ceramic body has a fracture toughness of greater than 5, and the mol % is based on the total moles of the yttria-stabilized zirconia at the respective location. In certain examples of this embodiment, the sintered yttria-stabilized zirconia ceramic body has a fracture toughness of greater than 6.8. In certain examples of this embodiment, 100% of the localized yttria concentrations have a yttria concentration of less than 7.5 mol %. In certain examples of this embodiment, the ceramic body has a bulk yttria concentration of 3.2 mol % to 3.5 mol %. In certain examples of this embodiment, the ceramic body has a bulk yttria concentration of 3.6 mol % to 4 mol %.

Another embodiment is a sintered yttria-stabilized zirconia ceramic body having a composite system with different localized yttria concentrations within the composite system, wherein more than 30% of the localized yttria concentrations have a yttria concentration of less than 2.5 mol %, less than 50% of the localized yttria concentrations have a yttria concentration greater than 5.8 mol %, less than 50% of the localized yttria concentrations have a yttria concentration greater than 6.5 mol %, and at least 35% of the localized yttria concentrations have a yttria concentration of between 6 and 7.5 mol % wherein the sintered yttria-stabilized zirconia body has a fracture toughness of greater than 4, and the mol % is based on the total moles of the yttria-stabilized zirconia at the respective location. In certain examples of this embodiment, 100% of the localized yttria concentrations have a yttria concentration of less than 7.5 mol %. In certain examples of this embodiment, the ceramic body has a bulk yttria concentration of 4 mol % to 4.7 mol %.

Another embodiment is a sintered yttria-stabilized zirconia ceramic body having a composite system with different localized yttria concentrations within the composite system, wherein more than 20% of the localized yttria concentrations have a yttria concentration of less than 2.5 mol %, less than 55% of the localized yttria concentrations have a yttria concentration greater than 5.8 mol %, less than 50% of the localized yttria concentrations have a yttria concentration greater than 6.5 mol %, and at least 40% of the localized yttria concentrations have a yttria concentration of between 6 and 7.5 mol % wherein the sintered yttria-stabilized zirconia body has a fracture toughness of greater than 3, and the mol % is based on the total moles of the yttria-stabilized zirconia at the respective location. In certain examples of this embodiment, 85% of the localized yttria concentrations have a yttria concentration of less than 7.5 mol %. In certain examples of this embodiment, the ceramic body has a bulk yttria concentration of 4.7 mol % to 5.5 mol %.

The two or more-phase composite system is produced by mixing two different YSZ powders having different yttria concentrations, forming the resulting mixture into a desired shape, and then sintering the shaped mixture resulting in a ceramic body. The two different powders are mixed together to provide a target bulk yttria concentration. For example, a first powder having a first yttria concentration is mixed with a second powder having a second yttria concentration, wherein the first yttria concentration is higher than the second yttria concentration.

In certain embodiments, the first yttria concentration is 4 to 8 mol % yttria and the second yttria concentration is 2 to 2.6 mol % yttria, based on the total weight of the first powder and the second powder, respectively. In certain embodiments, the second yttria concentration is 2 mol % yttria. The amount of the first powder may range from 5 to 70, particularly 5 to 61, more preferably 5 to 45, wt %, based on the total weight of the mixture. The amount of the second powder may range from 30 to 95, particularly 39 to 95, more preferably 55 to 95, wt %, based on the total weight of the mixture.

In certain embodiments, the first yttria concentration is 8 mol % yttria and the second yttria concentration is 2 mol % yttria.

In certain embodiments, the first yttria concentration is 5.3 mol % yttria and the second yttria concentration is 2 mol % yttria.

In certain embodiments, the first yttria concentration is 4 mol % yttria and the second yttria concentration is 2 mol % yttria.

Zirconia materials may comprise approximately 85 wt % to approximately 100 wt % zirconia or stabilized zirconia, based on the total weight of the zirconia ceramic material, or approximately 85 wt % or greater, or approximately 90 wt % or greater, or approximately 95 wt % or greater, or more than approximately 97 wt % or greater zirconia or stabilized zirconia, based on the total weight of the zirconia ceramic material.

Yttria-stabilized zirconia powders used as starting materials may, optionally, include a small amount of alumina (aluminum oxide, Al₂O₃) as an additive. For example, some commercially available yttria-stabilized zirconia ceramic materials include alumina at concentrations of from 0 wt % to 2 wt %, or from 0 wt % to 0.25 wt %, such as 0.1 wt %, relative to the zirconia material. Other optional additives of the powder starting material may include coloring agents to obtain shaded zirconia ceramic powder that may be formed by, for example, casting into shaded ceramic blocks that have a dentally acceptable shade or pre-shade upon sintering.

The YSZ powder mixture may be formed into a shape such as a block, disk, near net shape, or a form that approximates the size and/or shape of a single or multi-unit dental restoration, such as a crown, on-lay, bridge including a multi-unit bridge comprising restorations having more than one tooth structure, a partial or full solid-body denture, or a supporting structure such as an implant or an abutment. Porous ceramic bodies may be made from the YSZ powder mixtures, for example, by pressing, casting, or injection molding ceramic powders, or by automated additive (e.g., 3-D printing) and subtractive (e.g., milling) processes, including CAD and/or CAM processes. Processes include, but are not limited to, those described in commonly owned U.S. Pat. Nos. 9,365,459, 9,434,651, and 9,512,317, all of which are hereby incorporated in their entirety, herein.

Ceramic bodies prepared by the methods disclosed herein may be sintered in accordance with instructions of the manufacturer of commercially available ceramic bodies, or by heating at a temperature, for example, between about 1300° C. and 1600° C., for about 2 hours to 48 hours.

After sintering, the sintered bodies can be machined into a final patient-specific dental prostheses. The dental prostheses may be shaped into a crown, a multi-unit bridge, an inlay or onlay, a veneer, a full or partial denture, or other dental prosthesis.

In certain embodiments, the sintered body may be provided in the shape of a cylinder or cuboid block that may be loaded into a mill with an optional holder for attachment and milled chairside in a dental office to generate a dental article such as a crown, bridge, veneer, inlay or onlay that may be seated onto a patient.

### EXAMPLES

### Zirconia Powder Source:

Commercially available spray dried zirconia powders were used. The powders contained different yttria levels and powder size distribution as listed in Table 1.

The powder size distributions used are listed in Table 1.

TABLE 1
Powder size distribution of different powders

| | Diameter on cumulative % | | |
|---|---|---|---|
| Powders | 10% (um) | 50% (um) | 90% (um) |
| 2 Y | 24.22 | 44.71 | 74.12 |
| 3 Y | 32.3 | 49.5 | 76.5 |
| 4 Y | 33.53 | 52.45 | 66.68 |
| 5.3 Y | 34.49 | 52.36 | 81.62 |

### Mixing Condition:

The precursor powders from low yttria and high yttria systems can be mixed in different ratios using mixing apparatus such as a jar roller, V-blender or Munson mixer. The mixed powder should not be broken down or milled to maintain the scale of mixing at the granular level to >20 microns.

The different powder blends containing high yttria and low yttria levels were mixed in the ratio shown in Table 2 below.

### Green Body Formation:

The powders from high yttria and low yttria levels were uniaxially pressed to about 220 MPa.

### Pre-Sintering Condition:

These embodiments contain binder and may include a pre-sintering step for the removal of binder. For the binder included in the green body, a known binder used for molding of ceramics can be used, and an organic binder is preferable. Examples of such an organic binder include at least one selected from the group consisting of polyvinyl alcohol, polyvinyl butyrate, wax, and acrylic resin, preferably include at least one selected from the group consisting of polyvinyl alcohol and acrylic resin, and more preferably include acrylic resin. The green bodies were bisque fired to 950° C. to obtain bisque bodies.

### Sintering Condition:

The bisque bodies obtained by heat-treating the green bodies were sintered at conditions described below to obtain a sintered body.

The bisque bodies were first sintered to T1-1200° C. at heating rate of 600° C./hr and dwelled for 1 hour followed by heating further up to T2-1300° C. at heating rate of 120° C./hr. Finally, it was further heated up to T3-1580° C. and dwelled for 2 hours and 30 min and cooled down.

### Example 1

In one embodiment, 83% of low yttria powder (2Y) was mixed with 17% of high yttria powder (8Y). The final yttria level of the mix was targeted to contain 3 mol % YSZ. The mixed powder systems were sintered as described above and Table 2 shows evaluation results of the sintered bodies.

### Examples 2 to 10

Yttria stabilized zirconia powders according to Example 2 to 10 were mixed similar to Example 1 except the ratio of high yttria and low yttria powders were altered to reach the targeted final yttria level. The “powder type” for low yttria was 2Y for all the examples but the “powder type” for high yttria differed. The wt % and yttria level of different powder blends are listed in Table 2.

Table 2 below shows the results of fracture toughness for mixed powder system containing low yttria powder (2Y) and high yttria powder (8Y or 5.3Y or 4Y).

| | | | | | | Fracture | | | | |
|---|---|---|---|---|---|---|---|---|---|---|
| | High | | Low | | | Toughness | EDS - Yttria | | | |
| | Yttria | % High | Yttria | % Low | mol % | Fracture | distribution | | Crack length (μm) | |
| Ex. | powder | Y | powder | Y | Y | Toughness _{(HV-2)} | Y % < 2.5 | Y % < 2.8 | <10 μm | <20 μm |
| Ex-1 | 8Y | 17 | 2Y | 83 | 3.0 | 8.4 | 83.3% | 86.7% | 85.0% | 92.0% |
| Ex-2 | 8Y | 25 | 2Y | 75 | 3.5 | 7.2 | 53.3% | 56.7% | 65.0% | 80.0% |
| Ex-3 | 8Y | 34 | 2Y | 66 | 4.0 | 5.8 | 51.7% | 53.3% | 47.5% | 62.5% |
| Ex-4 | 8Y | 45 | 2Y | 55 | 4.7 | 4.6 | 30.0% | 40.0% | 30.2% | 47.9% |
| Ex-5 | 8Y | 55 | 2Y | 45 | 5.3 | 3.7 | 20.0% | 41.67% | 15.00% | 30.00% |
| Ex-6 | 5.3Y | 30 | 2Y | 70 | 3.0 | 8.9 | 56.7% | 70.0% | 78.8% | 92.5% |
| Ex-7 | 5.3Y | 45 | 2Y | 55 | 3.5 | 7.0 | 46.7% | 66.7% | 57.5% | 81.3% |
| Ex-8 | 5.3Y | 61 | 2Y | 39 | 4.0 | 5.7 | 23.3% | 33.3% | 40.0% | 58.8% |
| Ex-9 | 5.3Y | 70 | 2Y | 30 | 4.3 | 4.5 | 13.3% | 16.7% | 25.0% | 41.3% |
| Ex-10 | 4Y | 45 | 2Y | 55 | 3.0 | 8.3 | 56.7% | 63.3% | 80.0% | 96.3% |

Table 3 shows the powder size distribution for mixed powder system containing low yttria powder (2Y) and high yttria powder (8Y or 5.3Y or 4Y)

| | High | | Low | | | Diameter on cumulative % | | |
|---|---|---|---|---|---|---|---|---|
| | Yttria | % High | Yttria | % Low | mol % | 10% | 50% | 90% |
| Ex | powder | Y | powder | Y | Y | (μm) | (μm) | (μm) |
| Ex-1 | 8Y | 17 | 2Y | 83 | 3.0 | | | |
| Ex-2 | 8Y | 25 | 2Y | 75 | 3.5 | 28 | 49 | 81 |
| Ex-3 | 8Y | 34 | 2Y | 66 | 4.0 | | | |
| Ex-4 | 8Y | 45 | 2Y | 55 | 4.7 | | | |
| Ex-5 | 8Y | 55 | 2Y | 55 | 5.3 | | | |
| Ex-6 | 5.3Y | 30 | 2Y | 70 | 3.0 | 27 | 47 | 76 |
| Ex-7 | 5.3Y | 45 | 2Y | 55 | 3.5 | | | |
| Ex-8 | 5.3Y | 61 | 2Y | 39 | 4.0 | 30 | 49 | 79 |
| Ex-9 | 5.3Y | 70 | 2Y | 30 | 4.3 | | | |
| Ex-10 | 4Y | 45 | 2Y | 55 | 3.0 | 28 | 48 | 71 |

### COMPARATIVE EXAMPLES

In Comparative Examples 11 to 22 the powder blends consisted of low yttria powder (3Y) mixed with high yttria powder (4Y or 5.3Y or 8Y) to target yttria level between 3Y-5.6Y. The mixed powder systems were sintered as described above and Table 4 shows evaluation results of the sintered bodies.

Table 4 below shows the results of fracture toughness for mixed powder system containing low yttria powder (3Y) and high yttria powder (8Y or 5.3Y or 4Y)

| | | | | | | Fracture | | | | |
|---|---|---|---|---|---|---|---|---|---|---|
| | High | | Low | | | Toughness | EDS - Yttria | | | |
| | Yttria | % High | Yttria | % Low | mol % | Fracture | distribution | | Crack length (μm) | |
| Ex. | powder | Y | powder | Y | Y | Toughness _{(HV-2)} | Y % < 2.5 | Y % < 2.8 | <10 μm | <20 μm |
| CEx-11 | 8Y | 10 | 3Y | 90 | 3.5 | 4.2 | 0.0% | 3.4% | | |
| CEx-12 | 8Y | 20 | 3Y | 80 | 4.0 | 3.9 | 0.0% | 1.7% | 0.0% | 10.0% |
| CEx-13 | 8Y | 34 | 3Y | 66 | 4.7 | 3.5 | 0.0% | 0.0% | 0.0% | 0.0% |
| CEx-14 | 8Y | 45 | 3Y | 55 | 5.3 | 3.1 | 0.0% | 0.0% | 0.0% | 0.0% |
| CEx-15 | 8Y | 52 | 3Y | 48 | 5.6 | 3.1 | 0.0% | 0.0% | 0.0% | 10.0% |
| CEx-16 | 5.3Y | 44 | 3Y | 56 | 4.0 | 4.1 | 0.0% | 0.0% | 0.0% | 26.3% |
| CEx-17 | 5.3Y | 70 | 3Y | 30 | 4.6 | 3.3 | 0.0% | 0.0% | 0.0% | 3.8% |
| CEx-18 | 5.3Y | 80 | 3Y | 20 | 4.8 | 3.1 | 0.0% | 0.0% | 0.0% | 1.3% |
| CEx-19 | 5.3Y | 90 | 3Y | 10 | 5.1 | 3.0 | 0.0% | 0.0% | 0.0% | 5.0% |
| CEx-20 | 4Y | 70 | 3Y | 30 | 3.7 | 4.1 | 0.0% | 3.3% | 0.0% | 10.0% |
| CEx-21 | 4Y | 80 | 3Y | 20 | 3.8 | 4.0 | 0.0% | 0.0% | 0.0% | 7.5% |
| CEx-22 | 4Y | 90 | 3Y | 10 | 3.9 | 3.9 | 1.7% | 1.7% | 0.0% | 1.3% |

In Comparative Examples 23 to 25 the mixing of different powders were at the size of primary particles ˜300 nm or at average particles sizes less than 1.5 micron as shown in Table 5 below.

Table 5 below shows average particle size distribution for examples 23 to 25

| | High | | Low | | | Diameter on cumulative % | | |
|---|---|---|---|---|---|---|---|---|
| | Yttria | % High | Yttria | % Low | mol % | 10% | 50% | 90% |
| Ex. | powder | Y | powder | Y | Y | (μm) | (μm) | (μm) |
| CEx-23 | 8Y | 20 | 3Y | 80 | 4.0 | 0.1 | 0.15 | 0.2 |
| CEx-24 | 8Y | 26 | 2.6Y | 74 | 4.0 | 0.2 | 0.4 | 1 |
| CEx-25 | 8Y | 37.5 | 0Y | 62.5 | 3.0 | 0.2 | 0.4 | 1.2 |
| CEx-26 | 3Y | 100 | N/A | N/A | 3.0 | | | |
| CEx-27 | 4Y | 100 | N/A | N/A | 4.0 | | | |
| CEx-28 | 5.3Y | 100 | N/A | N/A | 5.3 | | | |

### Comparative Example 23

In another embodiment, 80% of low yttria powder (3Y) was mixed with 20% of high yttria powder (8Y). The final yttria level of the mix was targeted to contain 4 mol % YSZ. The mixed powder systems were sintered as described above and Table 6 shows evaluation results of the sintered bodies.

### Comparative Example 24

In another embodiment, 74% of low yttria powder (2.6Y) was mixed with 26% of high yttria powder (8Y). The final yttria level of the mix was targeted to contain 4 mol % YSZ. The mixed powder systems were sintered as described above and Table 6 shows evaluation results of the sintered bodies.

### Comparative Example 25

In another embodiment, 62.5% of low yttria powder (0Y) was mixed with 37.5% of high yttria powder (8Y). The final yttria level of the mix was targeted to contain 3 mol % YSZ. The mixed powder systems were sintered as described above and Table 6 shows evaluation results of the sintered bodies.

Comparative Examples 26-28 shows sintered bodies obtained from single source yttria system. The mixed powder systems were sintered as described above and Table 6 shows evaluation results of sintered bodies.

Table 6 below shows the results of fracture toughness for mixed powder system containing low yttria powder (0Y or 2.6Y or 3Y) and high yttria powder (8Y). The powders obtained from single source targeting 3Y, 4Y and 5.3Y are recorded below.

| | | | | | | Fracture | | | | |
|---|---|---|---|---|---|---|---|---|---|---|
| | High | | Low | | | Toughness | EDS - Yttria | | | |
| | Yttria | % High | Yttria | % Low | mol % | Fracture | distribution | | Crack length (μm) | |
| Ex. | powder | Y | powder | Y | Y | Toughness _{(HV-2)} | Y % < 2.5 | Y % < 2.8 | <10 μm | <20 μm |
| CEx-23 | 8Y | 20 | 3Y | 80 | 4.0 | 3.8 | 0.0% | 0.0% | | |
| CEx-24 | 8Y | 26 | 2.6Y | 74 | 4.0 | 4.4 | 0.0% | 11.7% | | |
| CEx-25 | 8Y | 37.5 | 0Y | 62.5 | 3.0 | 5.9 | 5.0% | 28.3% | | |
| CEx-26 | 3Y | 100 | N/A | N/A | 3.0 | 5.0 | 0.0% | 13.8% | 0.0% | 80.0% |
| CEx-27 | 4Y | 100 | N/A | N/A | 4.0 | 3.5 | 0.0% | 0.0% | 0.0% | 0.0% |
| CEx-28 | 5.3Y | 100 | N/A | N/A | 5.3 | 2.5 | 0.0% | 0.0% | 0.0% | 0.0% |

### Test Methods:

### Polishing Procedure:

The sintered zirconia samples were first cross-sectioned and then mounted in epoxy resin. The samples were polished to obtain a scratch-free surface according to the polishing steps of Table 7 below. The samples after polishing were ready for SEM, EDS and Fracture toughness analysis.

TABLE 7
Polishing procedure for the samples

| | Grinding | | Force | Head Speed | Plate Speed | Duration |
|---|---|---|---|---|---|---|
| Step # | Grit | Polishing Media | (lbs.) | (rpm) | (rpm) | (min) |
| 1 | 80 | — | 30 | 80 | 120 | 0.5-1 |
| 2 | 220 | — | 30 | 80 | 120 | 1 |
| 3 | 500 | — | 30 | 80 | 120 | 7 |
| 4 | 1200 | — | 30 | 80 | 120 | 12 |
| 5 | — | 15 μm diamond | 30 | 80 | 120 | 15 |
| | | suspension | | | | |
| 6 | — | 3 μm diamond | 30 | 80 | 120 | 15 |
| | | suspension | | | | |
| 7 | — | 1 μm diamond | 30 | 80 | 120 | 15 |
| | | suspension | | | | |
| 8 | — | 0.06 μm Silica | 30 | 80 | 120 | 15 |
| | | Suspension | | | | |

### Energy Dispersive X-Ray Spectroscopy

The elemental composition of sintered zirconia ceramic bodies was measured by Energy Dispersive X-ray (EDX), (QUANTAX 75, BRUKER), using the focused electron beam in the scanning electron microscope (SEM, TM3030 Plus, Hitachi, Japan).

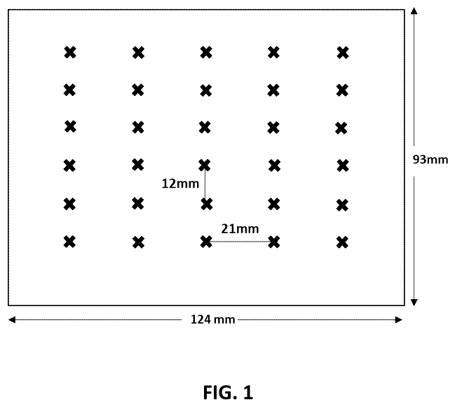

EDS spot analysis was performed on samples polished using procedure given in Table 7. In order to select the spots a pre-marked sheet (as shown in FIG. 1) was used as a guide. The pre-marked sheet had length of 124 mm and height of 93 mm and the aspect ratio was 1.3. The vertical distance between two marked location on the pre-market sheet were ˜12 mm and horizontal distance between two marked location on the pre-market sheet were ˜21 mm. The size of the pre-marked sheet was equivalent to size of image on the EDS testing window.

To ensure the spot location was the same irrespective of the sample analyzed, the pre-marked sheet was placed over the EDS image and spots were selected at the location same as on the pre-marked sheet. EDS was collected from 30 spots per SEM micrograph. For each sample, two SEM micrographs (magnification ×200) were used for EDS analysis. Hence, 60 points in total were collected for each sample to obtain the yttria distribution.

The measurement conditions were as follows:

Energy resolution Copper: ≤eV FWHM at Cu Kα; working distance: 7-9 mm; ICR detection: 5-18 kcps; accelerated voltage: 15 kV; scanning mode: exhaust, spot analysis; magnification: ×300 selected elements: Zr, Y, Hf, C, N, O. Each spot has the spatial resolution of 2 microns.

### Yttria Distribution Analysis:

The values quantified via EDX are normalized to Y/Zr and the yttria mol % were calculated using equation:

$$[ Y / Zr \% ] = 0.0241 [ Y mol \% ] - 0.0064$$

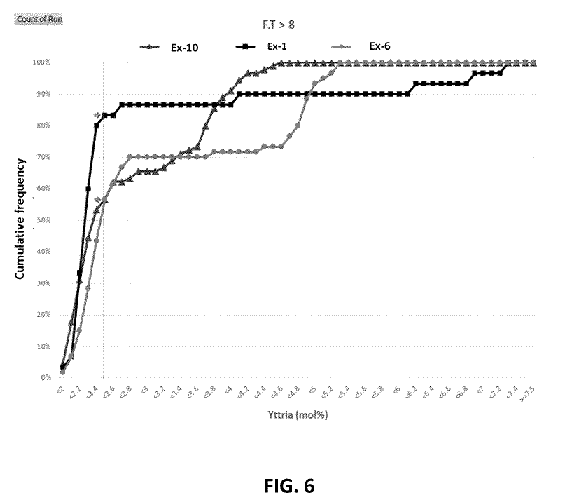

The cumulative frequency of distribution of yttria level in each mixed systems was determined and correlated to fracture toughness values (FIGS. 6 &7)

### Fracture Toughness Analysis:

Fracture toughness testing was performed on sintered and polished ceramic bodies using a Shimadzu Micro Hardness Tester (HMV-G21) testing machine with a Vickers indenter fixture. The polishing procedure is listed in Table 7. The measurements were taken from 20 random spots on each sample to obtain a more accurate representation of fracture toughness values for composite systems.

The method of testing fracture toughness was based on Brian Lawn's calculation (1980) and G. R. Anstis (J. Am. Ceram. Soc., 64(9), P533-538, 1981).

### Using the Equation:

$${K}_{IC} = 0.0205 * [ 2 √ ( E / H ) ] * [ P / ( 3 / 2 √ C ) ]$$

Wherein K_{ic}: Fracture Toughness (MPa·m^{-1/2}); E: Young's modulus (GPa); H: Vickers hardness (GPa)*, calculated by

$$H = 1.854 * ( P / {d}^{2} )$$

Wherein P applied load (N); C: crack length from the center of the impression to the crack tip; d: the length of the indentation diagonal. (Hardness Measurement Ref.: Vander, G. F. (2000). Microindentation hardness testing according to H. Kuhn & D. Medlin (Eds.), ASM Handbook, Volume 8: Mechanical Testing and Evaluation (pp. 221-231). ASM International.)

### Crack Length Distribution:

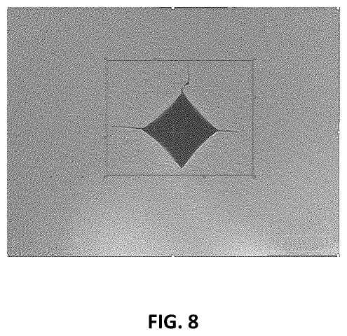

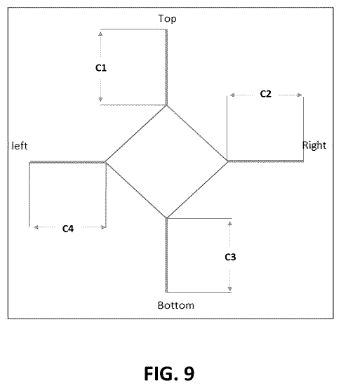

In order to accurately observe the endpoint of each crack, the length of the crack was measured using Zeiss optical microscope. They were determined by measuring the length from the indent vertices to the end of the Palmquist crack. Since the crack propagation for mixed powder system were not equal in each direction (FIG. 8), the measurement of crack length in each direction was recorded separately (as depicted in FIG. 9).

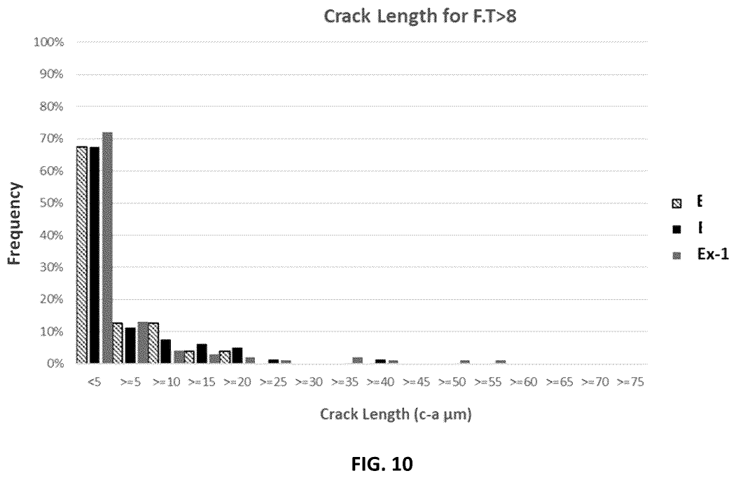

Each indent gave 4 set of data; C1, C2, C3 and C4 respectively. For 20 indents, about 80 data points of crack lengths were recorded. The frequency distribution of crack length per samples were statically analyzed and correlated to fracture toughness values (FIGS. 10 & 11)

### Particle Size Analysis

The spray-dried powder size distribution was measured using HORIBA (Laser Scattering Particle Size Distribution Analyzer LA-960). The measurement conditions for feeder were 65-100 and air was 0.01 MPa.

In view of the many possible embodiments to which the principles of the disclosed invention may be applied, it should be recognized that the illustrated embodiments are only preferred examples of the invention and should not be taken as limiting the scope of the invention.

What is claimed is:

1. A sintered yttria-stabilized zirconia ceramic body comprising a composite system with different localized yttria concentrations within the composite system, wherein more than 50% of the localized yttria concentrations have a yttria concentration of less than 2.5 mol %, more than 55% of the localized yttria concentrations have a yttria concentration of less than 2.8 mol %, less than 40% of the localized yttria concentrations have a yttria concentration greater than 5.8 mol %, less than 30% of the localized yttria concentrations have a yttria concentration greater than 6.5 mol %, and at least 30% of the localized yttria concentrations have a yttria concentration of between 6 and 7.5 mol %, wherein the sintered yttria-stabilized zirconia ceramic body has a fracture toughness of greater than 5, and the wt % is based on the total weight of the yttria-stabilized zirconia at the respective location.

2. The ceramic body of claim 1, wherein the sintered yttria-stabilized zirconia ceramic body has a fracture toughness of greater than 6.8.

3. The ceramic body of claim 1, wherein 100% of the localized yttria concentrations have a yttria concentration of less than 7.5 mol %.

4. The ceramic body of claim 2, wherein 100% of the localized yttria concentrations have a yttria concentration of less than 7.5 mol %.

5. The ceramic body of claim 1, wherein the ceramic body has a bulk yttria concentration of 3.2 wt % to 3.5 mol %.

6. The ceramic body of claim 2, wherein the ceramic body has a bulk yttria concentration of 3.6 wt % to 4 mol %.

7. The ceramic body of claim 1, wherein the ceramic body is a dental prosthetic device.

8. The ceramic body of claim 2, wherein the ceramic body is a dental prosthetic device.

9. A sintered yttria-stabilized zirconia ceramic body comprising a composite system with different localized yttria concentrations within the composite system, wherein more than 30% of the localized yttria concentrations have a yttria concentration of less than 2.5 mol %, less than 50% of the localized yttria concentrations have a yttria concentration greater than 5.8 mol %, less than 50% of the localized yttria concentrations have a yttria concentration greater than 6.5 mol %, and at least 35% of the localized yttria concentrations have a yttria concentration of between 6 and 7.5 mol % wherein the sintered yttria-stabilized zirconia body has a fracture toughness of greater than 4, and the mol % is based on the total moles of the yttria-stabilized zirconia at the respective location.

10. The ceramic body of claim 9, wherein 100% of the localized yttria concentrations have a yttria concentration of less than 7.5 mol %.

11. The ceramic body of claim 9, wherein the ceramic body has a bulk yttria concentration of 4 mol % to 4.7 mol %.

12. The ceramic body of claim 10, wherein the ceramic body has a bulk yttria concentration of 4 mol % to 4.7 mol %.

13. The ceramic body of claim 9, wherein the ceramic body is a dental prosthetic device.

14. The ceramic body of claim 10, wherein the ceramic body is a dental prosthetic device.

15. A method comprising:
mixing a first yttria-stabilized zirconia powder having a yttria concentration of 4 to 8 mol % yttria, based on the total weight of the first powder, with a second yttria-stabilized zirconia powder having a yttria concentration of 2 to 2.6 mol % yttria, based on the total weight of the second powder;
forming the resulting mixture into a desired shape, and
sintering the shaped mixture resulting in a yttria-stabilized zirconia ceramic body.

16. The method of claim 15, wherein the second yttria-stabilized zirconia powder has a yttria concentration of 2 mol % yttria.

17. The method of claim 15, wherein the first yttria-stabilized zirconia powder has a yttria concentration of 8 mol % yttria and the second yttria-stabilized zirconia powder has a yttria concentration is 2 mol % yttria.

18. The method of claim 15, wherein the first yttria-stabilized zirconia powder has a yttria concentration of 5.3 mol % yttria and the second yttria-stabilized zirconia powder has a yttria concentration is 2 mol % yttria.

19. The method of claim 15, wherein the first yttria-stabilized zirconia powder has a yttria concentration of 4 mol % yttria and the second yttria-stabilized zirconia powder has a yttria concentration is 2 mol % yttria.

20. The method of claim 15, wherein 5 to 70 wt % of the first yttria-stabilized zirconia powder is mixed with 30 to 95 wt % of the second yttria-stabilized zirconia powder, based on the total weight of the mixture.
